(19) United States
(12) Patent Application Publication
(10) Pub. No.: US 2021/0052774 A1
(43) Pub. Date: Feb. 25, 2021

(54) FIXED-SHAPE TISSUE MATRIX AND RELATED METHODS

(71) Applicant: LifeCell Corporation, Madison, NJ (US)

(72) Inventor: Marianne P. Edwards, Brick, NJ (US)

(21) Appl. No.: 16/997,239

(22) Filed: Aug. 19, 2020

Related U.S. Application Data
(60) Provisional application No. 62/889,654, filed on Aug. 21, 2019.

(51) Int. Cl. A61L 27/36; A61F 2/12

(52) U.S. Cl. CPC A61L 27/3633; A61F 2/12; A61F 2002/526; A61L 27/3691; A61L 27/362

(57) ABSTRACT

The present disclosure provides tissue products produced from a combination of three-dimensional biologic scaffolds and acellular tissue matrices. The tissue products may include features for suture or fixation reinforcement. The tissue products may harness various properties of different acellular tissue matrices and different three-dimensional biologic scaffolds to provide improved composite structures with desired mechanical and/or biologic properties.

This application claims priority under 35 U.S.C. § 119 to U.S. Provisional Application No. 62/889,654, filed Aug. 21, 2019, the entire contents of which is incorporated herein by reference.

The present disclosure relates to tissue products, and, more particularly, to devices and methods to form tissue products into three-dimensional fixed shapes for use in reconstructive surgery.

Biologically derived acellular tissue scaffolds are engineered to achieve many goals, including facilitating the incorporation of the acellular tissue scaffold into the recipient tissue and promote regeneration of functional tissue. The ability of tissue scaffolds to incorporate and promote tissue regeneration creates many clinical applications for these compositions. For example, acellular tissue scaffolds may be used to promote regeneration of tissue lost to trauma, infection, ischemia, surgical resection of malignancy, and other causes. Acellular tissue scaffolds also have utility in aesthetic treatments and surgeries, including treatment of wrinkles, breast reconstruction or augmentation, and other tissue-augmentation procedures that may require removal of tissue.

Tissue removal, including removal of dermal and adipose tissues, is required for surgical treatment of cancer. Such tissue removal may result in an undesired external appearance or deformity, depending on the anatomic location affected. For example, during mastectomy for breast cancer, it is often necessary to remove the nipple along with the breast and other tissue to minimize the risk of a local recurrence of the cancer. In some cases, a patient may elect to undergo a breast reconstruction, either during the mastectomy operation or at a future time following healing from the surgery and completion of adjuvant therapy, such as chemotherapy and/or radiation. Breast reconstruction may utilize the patient's own skin, muscle, and adipose tissue to re-create the shape and volume of the missing breast, may involve insertion of an synthetic implant, such as a breast implant, or may include a combination of these procedures.

There are several available options for re-creating the appearance of a nipple on a reconstructed breast. A nipple for the reconstructed breast may be an external prosthesis or be surgically formed using the patient's tissue. A prosthetic nipple is temporarily secured to the skin and has the advantage of being customized, typically from a silicone-based material, in a shape and size to very closely match the patient's contralateral nipple, thus providing symmetry. Prosthetic nipples, however, are external appliances that are attached to the skin with a temporary adhesive and are immediately perceived as artificial when touched. Similar to other external prosthetic devices, a prosthetic nipple will wear out over time.

Surgically reconstructed nipples have the advantage of being permanent and having the feel of natural tissue. There are significant challenges in surgical nipple reconstruction, however, including matching the position, size, shape, and projection of the surgically reconstructed nipple with that of the contralateral nipple. Creating the proper shape, size, and projection (outward from the surrounding skin and areola) is imprecise, regardless of the skill of the surgeon. Donor skin for a nipple reconstruction is often harvested from the upper-inner thigh, creating a second incision which can be painful. Also, nipple and areola reconstruction is generally performed as a second procedure after 3-4 months to allow healing of the skin overlying the initial breast reconstruction.

Accordingly, a device and method for reconstructing a dermal appendage, such as a nipple, that provides for matching shape, size, and other physical characteristics of the natural structure of the appendage without the need for an external prosthesis is needed.

As described herein, the use of molded tissue products may be used to reconstruct a dermal appendage, such as a breast nipple-areolar complex, which is discussed by way of example. There are additional applications for such tissue products, wherein a tissue product having a custom-designed size and shape for a particular application in a particular patient is advantageous. Some non-limiting example applications include repair of abdominal wall and diaphragmatic hernias, sealing of fistulae, reconstruction of facial features, and others.

Regarding breast reconstruction, the nipple-areolar complex (“nipple”) can be imaged and tissue products can be molded to match the size and complex shape of a patient's own natural nipple. The contralateral nipple may be used as a “model” following unilateral mastectomy. Alternatively, one or both nipples can be imaged before a mastectomy or related breast resection procedure for use in designing a mold for a tissue product having a simple or complex shape. Techniques for designing and forming a mold-including a mold customized to a particular patient, are described, as is molding a tissue product, fixing the molded tissue product into a stable three-dimensional shape, and examples of implanting the completed tissue product in conjunction with a breast reconstruction procedure are disclosed herein. Methods of packaging and storing the molded tissue product prior to implantation are also disclosed.

Disclosed is a tissue product including a plurality of acellular tissue matrix particles formed into a three-dimensional shape. The three-dimensional shape is in the shape of an anatomic structure. The three-dimensional shape is substantially fixed by irradiation.

In some embodiments, the acellular tissue matrix particles include acellular dermal matrix. In some embodiments, the acellular tissue matrix particles include acellular adipose matrix. In some embodiments, the acellular tissue matrix particles include porcine-derived tissue matrix. In some embodiments, the acellular tissue matrix particles include human-derived tissue matrix. In some embodiments, the three-dimensional shape is formed by molding, electron beam exposure, dehydrothermal therapy, or a combination thereof. In some embodiments, the three-dimensional shape includes at least a first shape and a second shape. In some embodiments, the anatomical structure comprises a nipple. In some embodiments, the anatomic structure replicates an anatomic structure of a specific individual.

Also disclosed is a method of forming a tissue product with a fixed three-dimensional shape. The method includes forming a plurality of acellular tissue matrix particles to a mold with a three-dimensional shape, wherein the three-dimensional shape is in the shape of an anatomic structure. The plurality of acellular tissue matrix particles are shaped within the mold into a stable, three-dimensional structure. The structure of the shaped plurality of acellular tissue matrix particles is substantially fixed with irradiation.

In some embodiments, the acellular tissue matrix particles include acellular dermal matrix. In some embodiments, the acellular dermal matrix particles comprise human acellular dermal matrix. In some embodiments, the acellular dermal matrix particles comprise porcine acellular dermal matrix. In some embodiments, the anatomic structure comprises a nipple. In some embodiments, the mold is a reverse mold of the anatomic structure. In some embodiments, the mold is a one-piece mold. In some embodiments, the mold is at least a two-piece mold. In some embodiments, the forming step includes injecting the particles into the mold. In some embodiments, the structure of the particles is substantially fixed with electron beam radiation. In some embodiments, the particles contained within the mold are treated with dehydrothermal therapy.

Also disclosed is a method of reconstructing a nipple. The method includes identifying a tissue site in need of reconstruction. The tissue site includes, or formerly included, a nipple. The method includes adding acellular tissue matrix particles to a mold comprising a three-dimensional shape. The three-dimensional shape corresponds to a desired nipple shape. The acellular tissue matrix particles are shaped within the mold into the three-dimensional shape to form a tissue product. The structure of the tissue product is substantially fixed with irradiation. The tissue product is removed from the mold. The tissue product is implanted within or on the tissue site.

In some embodiments, the acellular tissue matrix particles include acellular dermal matrix. In some embodiments, the acellular dermal matrix particles comprise human acellular dermal matrix. In some embodiments, the acellular dermal matrix particles comprise porcine acellular dermal matrix. In some embodiments, the mold is a reverse of the tissue site. In some embodiments, the mold is a one-piece mold. In some embodiments, the mold is at least a two-piece mold. In some embodiments, the particles are injected into the mold. In some embodiments, the method further includes substantially fixing the structure of the tissue product with electron beam radiation. In some embodiments, the method further includes treating the particles contained within the mold with dehydrothermal therapy.

### BRIEF DESCRIPTION OF THE DRAWINGS

FIG. 1 is a top view of a mold for creating a three-dimensional tissue product according to some embodiments;

FIG. 2 is a top view the mold of FIG. 1 with tissue particles being added according to some embodiments;

FIG. 3 is a top view of a formed tissue product within the mold of FIG. 1 according to some embodiments;

FIG. 4 is a top view of a formed tissue product removed from the mold of FIG. 1 and arranged next to the mold according to some embodiments;

FIG. 5 is an illustration of a surgical procedure for insertion of a formed tissue product into a tissue site according to some embodiments; and

FIG. 6 is a side view of a formed tissue product according to some embodiments.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts. The accompanying drawings, which are incorporated in and constitute part of this specification, illustrate exemplary embodiments of the present disclosure. Together with the description, the drawings serve to explain the principles of the disclosure.

In this application, the use of the singular includes the plural unless specifically stated otherwise. In this disclosure, the use of “or” means “and/or” unless stated otherwise. Furthermore, the use of the term “including,” as well as other forms, such as “includes” and “included,” is not limiting. Any range described herein will be understood to include the endpoints and all values between the endpoints.

The section headings used herein are for organizational purposes only and are not to be construed as limiting the subject matter described. All documents or portions of documents cited in this disclosure, including but not limited to patents, patent applications, articles, books, and treatises, are hereby expressly incorporated by reference in their entirety for any purpose.

Various human and animal tissues can be used to produce products for treating patients. For example, tissue products for regeneration, repair, augmentation, reinforcement, and/or treatment of human tissues that have been damaged or lost due to various diseases and/or structural damage (e.g., from trauma, surgery, atrophy, degeneration) have been produced. Such products can include, for example, acellular tissue matrices, tissue allografts or xenografts, and/or reconstituted tissues (i.e., at least partially decellularized tissues that have been seeded with cells to produce viable materials). Grafting of a substantially acellular dermal tissue matrix (“ADM”) has been used in various clinical applications, including to replace supporting and structural soft tissue elements such as muscle fascia or gingival tissue, and for interposition between a biocompatible implant or tissue expander and the recipient host's tissues, for example.

A variety of tissue products have been produced for treating bone and soft tissues. For example, ALLODERM® and STRATTICE® (LIFECELL CORPORATION, Branchburg, N.J.) are two dermal acellular tissue matrices made from human and porcine dermis, respectively. Although such materials are very useful for treating certain types of conditions, materials having different biological and/or mechanical properties may be desirable for certain applications. For example, ALLODERM® and STRATTICE® have been used in the surgical treatment of structural defects and to provide support to tissues for abdominal walls or in breast reconstruction, for example. The mechanical and biological properties of tissue products make them well-suited for these and other uses, but they may not be ideally shaped for certain indications.

Reconstruction of the breast and other tissues may be accomplished using a tissue scaffold composed of partially or completely decellularized tissue. Tissue scaffolds having a three-dimensional component add volume and shape to the recipient's implantation site. Unlike synthetic implants, however, tissue scaffolds induce a minimal or absent host inflammatory response and induce regeneration of tissue at the site. Tissue scaffolds, such as extracellular tissue matrices, mimic the extracellular matrix of the surrounding native tissue into which the scaffold is implanted. This property of extracellular tissue matrices may favorably induce cells at the implantation site, such as fibroblasts, adipocytes, myocytes, and other cell types, to transform the implanted tissue scaffold into a desired tissue type.

Consequently, three-dimensional tissue scaffolds have potential for use in many clinical applications. In one example disclosed herein, a dermal appendage, such as a nipple-areolar complex, can be engineered from an acellular tissue matrix, molded into a three-dimensional shape, fixed to retain the shape and volume, and used in a surgical breast reconstruction procedure. Other examples of creating a fixed, stable three-dimensional tissue matrix form are also discussed, including creating custom-engineered tissue forms such as conical forms to fill fistulae. Methods of forming fixed three-dimensional tissue matrix shapes for use in other soft-tissue rehabilitative or aesthetic reconstruction procedures are provided.

Use of a flowable acellular tissue product in a nipple mold provides many benefits. For example, the molded tissue product may produce symmetrical nipples in a variety of sizes. The molds can be 3-D printed to provide for specific shapes and sizes. The molded tissue product may be implanted as part of a single-stage breast reconstruction procedure. Further, the mold could double as a first layer sterile barrier inside an outer packaging of the tissue product.

As used herein, “tissue product” will refer to any human or acellular tissue that contains an extracellular matrix protein. “Tissue products” may include acellular or partially decellularized tissue matrices, as well as decellularized tissue matrices that have been repopulated with exogenous cells.

As used herein, the term “acellular tissue matrix” will refer to an extracellular matrix derived from human or animal tissue, wherein the matrix retains a substantial amount of natural collagen and glycoproteins needed to serve as a scaffold to support tissue regeneration. “Acellular tissue matrixes” are different from purified collagen materials, such as acid-extracted purified collagen, which are substantially void of other matrix proteins and do not retain the natural micro-structural features of tissue matrix due to the purification processes. Although referred to as “acellular tissue matrixes,” it will be understood that such tissue matrixes may combine with exogenous cells, including, for example, stem cells or cells from a recipient in whom the “acellular tissue matrices” may be implanted. Further, it should be appreciated that “acellular tissue matrix particles” refer to a particulate of an acellular tissue matrix.

“Acellular” or “decellularized” tissue matrices will be understood to refer to tissue matrices wherein no cells are visible using light microscopy.

A “three-dimensional shape” or “3-D shape” relates to a shape having a length, a width, and a thickness. A rectangular solid, such as a cube, a cylinder, a sphere, and a cone are all non-limiting examples of three-dimensional shapes. Conversely, a sheet of material having a length or width of an order of magnitude, such as a 100×, a 1,000×, a 10,000×, or a greater than 10,000× order of magnitude than a thickness or depth is not a three-dimensional shape.

An “anatomic structure” is a structure relating to a body part of an animal or human. Some examples of an anatomic structure are a male or female nipple-areolar complex, an ear, a nose, a lip, a chin, a buccal fat pad, a breast; a muscle, such as a pectoral muscle, a deltoid muscle, a bicep muscle, a triceps muscle, a brachioradialis muscle, a gluteal muscle, a rectus femoris muscle, a biceps femoris muscle, a gastro-soleus muscle, and the like, without limitation; a fingertip; a tubular anatomic structure, such as a trachea, a bronchus, an esophagus, an entero-colonic tubular structure, an artery, a vein, a fallopian tube, a vas deferens, or another part of a body, without limitation.

“Substantially fixing” relates to setting a shape of a structure such that the structure retains its shape outside of a mold or other container. A structure that is substantially fixed may retain a level of pliability, elasticity, or sponge-like property. A structure that is substantially fixed is not entirely rigid. For example, a substantially fixed tissue product may be compressible and capable of expanding back to its substantially fixed shape after compression.

A “mold” relates to any three-dimensional structure possessing an open area configured to receive tissue matrix, pieces, particles, or whole tissue matrix. A mold may be pre-formed or constructed immediately prior to use. The mold may be one piece or may be composed of two or more joined segments. The mold may be composed of any sterilizable material.

Tissue Product Materials

Source tissues are used to create acellular tissue matrices used to form various moldable tissue matrix products and compositions. The acellular tissue matrix may originate from a human or an animal tissue matrix. Suitable tissue sources for an acellular tissue matrix may include allograft, autograft, or xenograft tissues. Human tissue may be obtained from cadavers. Additionally, human tissue may be obtained from live donors; i.e. autologous tissue.

Some examples of non-human tissue sources which may be used for xenograft tissue matrices include pig, cow, dog, cat, or other animals from domestic or wild sources and/or any other suitable mammalian or non-mammalian xenograft tissue source. In some exemplary embodiments, the acellular tissue matrix may originate from a source dermal matrix taken from an animal, such as a pig. In one exemplary embodiment, the source dermal matrix may comprise one or more layers of skin that have been removed from an animal. The size and shape of the source tissue matrix may be varied, according to known methods, to produce differing amounts of acellular tissue matrix particles affecting the pliability of a (non-flowable) sheet of acellular tissue matrix or the viscosity of a flowable acellular tissue matrix composition.

In some embodiments, a flowable porcine acellular dermal matrix tissue product is used. The flowable tissue product may include dry particles. In some embodiments, the flowable tissue product may include tissue matrix particles suspended in a carrier. In further embodiments, the carrier may include hyaluronic acid. In some embodiments, the flowable tissue product is injectable.

If porcine or other animal sources are used, the tissue may be further treated to remove antigenic components, such as 1,3-alpha-galactose moieties, which are present in pigs and other mammals, but not humans or certain other primates. In some embodiments, the tissue is obtained from animals that have been genetically modified to lack expression of antigenic moieties, such as 1,3-alpha-galactose, for example. See Xu, Hui, et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is hereby incorporated by reference in its entirety.

Acellular tissue matrices can provide a suitable tissue scaffold to allow cell ingrowth and tissue regeneration. Starting materials for forming a dermal appendage having a fixed three-dimensional shape include an acellular dermal matrix (“ADM”), in some embodiments. In some embodiments, the ADM is a porcine acellular dermal matrix (“pADM”). In some embodiments, the ADM is a human ADM. Other sources of ADM could be used, as previously mentioned. The starting ADM material should comprise substantially non-cross-linked collagen to allow infiltration with host cells, including fibroblasts and vascular elements. Regardless, some degree of collagen cross-linking results from substantially fixing the ADM into a final shape of the nipple, or other molded tissue product.

In some embodiments, starting materials for forming a dermal appendage having a fixed three-dimensional shape include an acellular adipose matrix or an acellular muscle matrix. In some embodiments, the starting materials include a composite matrix including a combination of acellular dermal matrix, acellular adipose matrix, and/or acellular muscle matrix. The acellular adipose matrix and the acellular muscle matrix may be sourced from porcine or human tissue.

To form the three-dimensional shape, a moldable tissue product may be used. In some embodiments, the moldable tissue product comprises a flowable tissue product composition. The flowable tissue product composition may be liquid, semi-liquid, or an accumulation of dry particulate and, therefore, flowable, injectable, and takes the shape of its container, such as a mold. For example, a space bounded by a mold inner surface having a three-dimensional shape is filled with the flowable tissue product composition, in an exemplary embodiment. The flowable tissue product composition may comprise a viscosity conducive to pouring, injecting, spreading, packing, or otherwise inserting into the mold wherein the flowable tissue product composition contacts the inner surface and assumes the three-dimensional shape of the inner surface. After molding and fixing, a flowable tissue product composition will substantially retain a shape and volume over time following implantation in a recipient, with acceptable biological responses that include minimal or no inflammation and maximum infiltration with the recipient's cells.

In some embodiments, the moldable tissue product may include acellular tissue matrix particles or pieces. The moldable acellular tissue matrix particles or pieces are formed by subjecting the source tissue matrix to mechanical and/or chemical processing steps. Mechanical processing generally removes undesired tissues and reduces the source tissue into smaller particles. For example, a sheet of acellular tissue matrix may be cut into pieces or shredded into particles. Mechanical processing may further include grinding, grating, freeze-drying, fracturing, or other processes to break apart tissue. The source tissue matrix may be checked for fatty tissue and cut to remove the tissue and/or to prevent tangling of tissue matrix pieces. The source tissue matrix may be frozen and thawed prior to mechanical processing.

Chemical processing is performed to reduce bio-burden and remove additional undesirable natural materials including a portion, or substantially all of, cells, lipid, and nucleic acids. For example, enzymes, detergents, and/or other agents may be used in one or more steps to further remove cellular materials or lipids, remove antigenic materials, and/or reduce the bacteria or other bioburden of the material. Chemical processing may also include washing with a protective storage solution. The tissue may be washed with phosphate-buffered saline (PBS) or detergents, such as sodium dodecyl sulfate or Triton®, to assist in cell and lipid removal. Enzymes such as lipases, DNAses, RNAses, alpha-galactosidase, alcalase, trypsin, bromelain, papain, ficin, or other enzymes may be used to ensure destruction of nuclear materials, antigens from xenogenic sources, residual cellular components and/or viruses. Further, acidic solutions and/or peroxides may be used to help further remove cellular materials and destroy bacteria and/or viruses, or other potentially infectious agents. In some embodiments, the source tissue is treated with peracetic acid.

Additional mechanical processing follows chemical processing, in some embodiments, to form the flowable acellular tissue matrix composition.

In some embodiments, the tissue matrix particles are sorted by size. In an exemplary embodiments, sequentially sized wire screens filter the particles into groups of particles within a similar size range.

Tissue Matrix Molds

To form a fixed shape tissue product for a soft tissue reconstruction application, such as nipple reconstruction, the tissue product is formed by molding the tissue matrix into a three-dimensional shape. In some embodiments, the three-dimensional shape corresponds to an anatomic structure. The anatomic structure may be a nipple-areolar complex.

The shape of a tissue product is formed in a mold that receives the flowable or pliable tissue matrix composition, applying a desired three-dimensional shape to the tissue matrix composition. The shape of the mold is modeled after a soft tissue anatomic structure, in some embodiments. In an exemplary embodiment, the shape is modeled after a shape of a nipple-areolar complex. This is not meant to be limiting, however; the creation of molds for forming a fixed shape tissue product in any of a great number of shapes useful for surgical reconstruction of soft tissues is possible. In some embodiments, the mold may be pre-formed to match a soft tissue anatomic structure from a particular patient. In some embodiments, the mold is formed into a standard size and shape, without consideration of a particular size and shape for a specific individual patient. The mold may be a one-piece open mold, a two-piece open mold, a two-piece injection mold, a multi-piece open or injection mold, or an alternative mold suitable for receiving a processed acellular tissue matrix, a flowable acellular tissue matrix composition, or an alternative form of tissue matrix product that is able to assume the shape if a container; i.e., the mold.

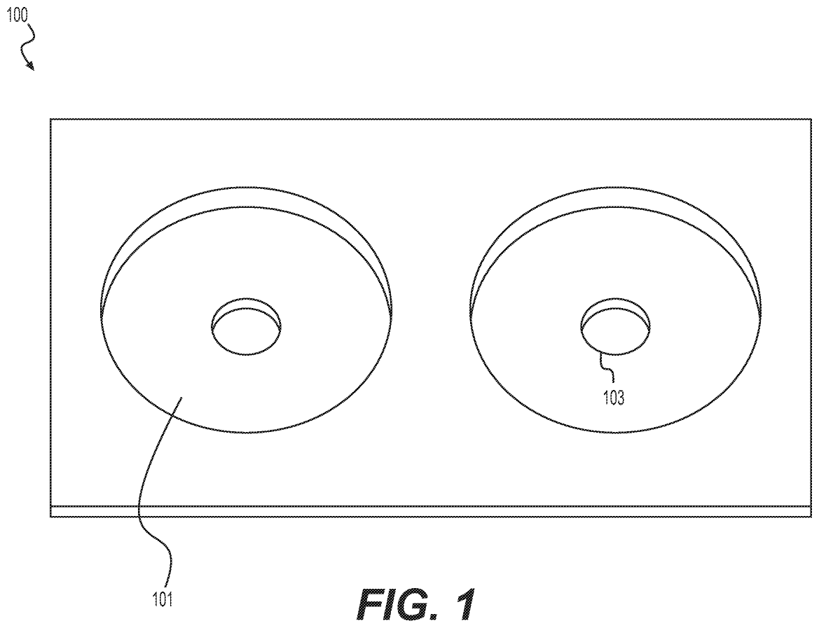

FIG. 1 depicts an exemplary mold 100 formed to shape acellular tissue matrix into the shape of a nipple-areolar complex. The mold 100 includes a top reservoir 101 and a bottom reservoir 103. In some embodiments, the top reservoir 101 corresponds to the areola and the bottom reservoir 103 corresponds to the nipple.

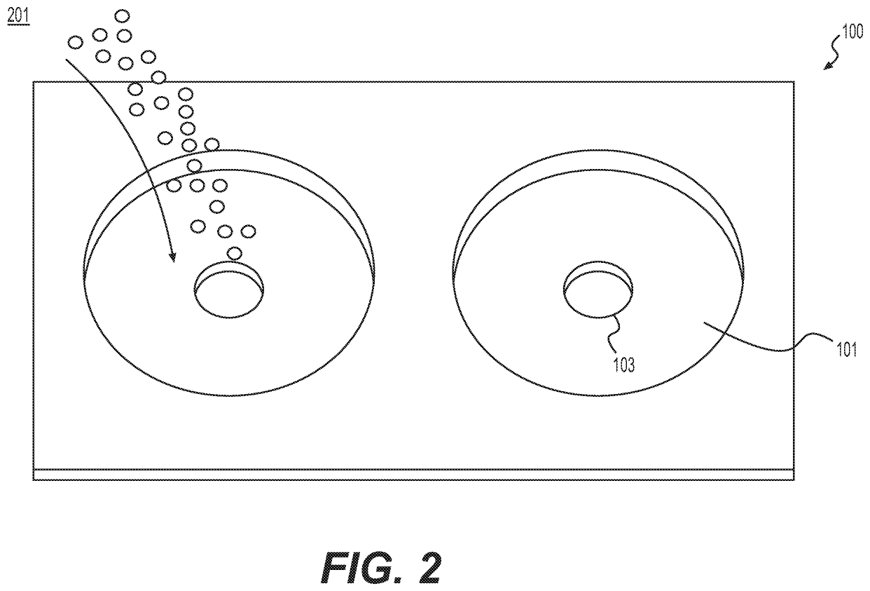

In some embodiments, the mold is filled with an acellular matrix as depicted in FIG. 2. In some embodiments, the acellular matrix is an ADM. In an exemplary embodiment, the acellular matrix is a porcine ADM. In a further embodiment, the acellular matrix is in the form of tissue matrix particles. In some embodiments, the particles are in the form of a flowable composition, i.e., in a carrier as discussed above. For example, particulate ADM may be poured into the mold 100. The particulate ADM fills into both bottom reservoir 103 and top reservoir 101. The porcine ADM may be combined with a flowable carrier, as discussed above, to form a flowable acellular tissue matrix composition.

In some embodiments, the mold 100 includes a shape of a nipple-areolar complex (“nipple”). The pre-formed nipple mold is filled 201 with the flowable acellular tissue matrix composition by pouring, spooning, scooping, injecting, spreading, or otherwise delivering the flowable composition into the mold 100. In some embodiments, the mold 100 comprises a one-piece body with a wide-based opening through which the mold 100 is completely filled with the acellular tissue matrix composition flush with a rim of the top reservoir 101 of the mold 100. In other embodiments, a portion of the acellular tissue matrix composition may be situated above the rim of the top reservoir 101 of the mold 100. After setting the shape of the acellular tissue matrix composition, the surface of the fixed tissue product at the opening forms a base of the fixed-shape tissue matrix product, such as the base of a fixed-shape tissue matrix product for reconstruction of a nipple-areolar complex, in some embodiments.

In some alternative embodiments, a two-piece mold may be used. A two piece mold may comprise a mold first piece and a mold second piece that articulate to form a cavity bearing the complex shape to be imparted to the molded tissue matrix product. In some embodiments, the mold first piece and the mold second piece are coupled, such as hingedly coupled, for example. In some embodiments, the mold first piece and the mold second piece articulate without coupling. In one exemplary embodiment, the mold first piece receives the acellular tissue matrix or flowable acellular tissue matrix composition. The mold second piece is then pressed into the tissue matrix or composition contained within the mold first piece, wherein the acellular tissue matrix or tissue matrix composition is biased into the inner surface of the mold first piece in response to pressure from the mold second piece, and wherein the tissue matrix assumes a shape of the inner surface.

In another exemplary embodiment, a pre-formed nipple mold is filled with a processed porcine acellular dermal matrix. The processed porcine acellular dermal matrix is mixed with water or other suitable fluid or hydrate the matrix. The hydrated matrix may be treated with additional solutions to affect the touch compression, pore size, alignment of collagen, and strength of adhesion forces holding the hydrated matrix together. In some embodiments, the additional solutions are the same solutions used to hydrate the matrix. In some embodiments, the water or other suitable fluid for hydration affects the touch compression, pore size, alignment of collagen, and strength of adhesion forces holding the hydrated matrix together.

The mold may be formed from various materials or combinations of materials suitable for forming molds and similar shaped objects, generally. Such materials include polymers, such as thermoplastics (including ABS, fluoropolymers, polyacetal, polyamide, polycarbonate, polyethylene, polysulfone, and/or the like); thermosets (including epoxy, phenolic resin, polyimide, polyurethane, silicone, and/or the like); glasses (including fiberglass); carbon-fiber, aramid-fiber, any combination thereof and/or like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any suitable material; and/or any combination thereof.

In some embodiments, the mold may be produced with 3D printing techniques. The shape of the mold may be determined from computerized tomography scans or other images taken of anatomic sites of a patient. For example, a patient may wish to receive tissue product to emulate a nipple-areolar complex in one breast. Images may be taken of the contralateral breast and a mold may be created with a shape corresponding to the nipple-areolar complex of the contralateral breast.

Substantial Fixation of the Tissue Product

After the acellular tissue matrix composition is contacted with the mold and assumes a three-dimensional shape of the mold, the acellular tissue matrix is substantially fixed to retain the three dimensional shape imparted by the mold. In some embodiments, the molded acellular tissue matrix is freeze-dried within the mold prior to fixation. In some embodiments, the molded acellular tissue matrix is freeze-dried within the mold following at least one fixation phase. In some embodiments, fixation is performed prior to freeze-drying of the acellular tissue matrix. Multiple factors affect the degree to which the three-dimensional shape of a molded acellular tissue matrix is fixed. Those factors include, but may not be limited to, the extent and type of collagen cross-linking within the molded acellular tissue matrix; the density, i.e., the dry weight of acellular tissue matrix particles per unit volume of the suspension or slurry of acellular tissue matrix particles delivered into the mold; and the chemical composition of the solution forming the suspension or slurry.

Substantial fixation of the three-dimensional shape may be affected by a degree of cross-linking between collagen molecules in two particles of acellular tissue matrix in contact with each other. A degree of cross-linking between collagen molecules of different particles is desirable to stabilize the three-dimensional shape. Extensive cross-linking between different particles, or cross-linking between collagen molecules of a single particle, however, is undesirable. Extensive collagen cross-linking may impede infiltration and colonization of the acellular tissue matrix with the recipient's cells, including fibroblasts and endothelial cells. Limited or delayed colonization with the recipient's cells delays, and may prevent, complete incorporation of the acellular tissue matrix into the recipient's tissues. Consequently, the acellular tissue matrix will comprise collagen cross-linking to a degree necessary to retain the three-dimensional shape, but not to a degree delaying or limiting incorporation of the acellular tissue matrix into the recipient's tissues.

As discussed above, the consistency (viscosity) and density (weight of dry acellular tissue matrix per unit volume of liquid used to form the suspension or slurry of acellular tissue matrix particles) is adjusted by changing the volume and composition of processing solutions used to form the suspension or slurry of acellular tissue matrix particles. In an exemplary embodiment, the percentage (dry weight per unit volume) of acellular tissue matrix within the suspension or slurry comprising the tissue product contacting the mold is between about ten percent (10%) to fifteen percent (15%). In some embodiments, the percentage is between about fifteen percent (15%) to twenty percent (20%). In some embodiments, the percentage is between about twelve percent (12%) to eighteen percent (18%). In some embodiments, the percentage is between about five percent (5%) to ten percent (10%).

In some embodiments, three-dimensional shape of the acellular tissue product contained within the mold is substantially fixed by exposing the tissue product to an electron beam. Electron beam exposure presumably affects the degree of collagen cross-linking. In an exemplary embodiment, the molded acellular tissue matrix is exposed to e-beam radiation at a dose between 10 kGy to 30 kGy. E-beam radiation may be applied while the acellular tissue matrix is in the mold after removal from the mold. In some embodiments, the acellular tissue matrix is placed in a foil package and sealed with a heat sealer prior to e-beam radiation. In further embodiments, the acellular tissue matrix is placed into the foil package while in the mold.

In some embodiments, the stabilization process involves other forms of irradiation besides electron-beam irradiation. Such other forms of irradiation may comprise ultraviolet light or gamma radiation.

In some embodiments, the stabilization process may include controlling the dose and type of irradiation to modulate various physical characteristics of the stabilized molded acellular tissue matrix product, such as tensile strength, elasticity, porosity, and stiffness. In some embodiments, controlling the dose and type of irradiation is employed to modulate resilience and volume retention in response to external compression by surrounding tissues after implantation of the tissue product into a recipient.

In some embodiments, the three dimensional shape of the acellular tissue product contained within the mold is substantially fixed by treating the tissue product to a dehydrothermal treatment, such as by heating the material in a vacuum. In some embodiments, the dehydrothermal treatment is performed after freeze-drying of the molded acellular tissue matrix. Dehydrothermal treatment is performed, in one exemplary embodiment, by heating the molded acellular tissue matrix in a vacuum to between about 70° C. to about 120° C. or between about 80° C. and about 110° C. or to about 100° C., or any values between the specified ranges in a reduced pressure or vacuum. As used herein, “reduced pressure” means a pressure at least about ten percent (10%) less than the standard atmospheric pressure of 760 mmHg.

In some embodiments, substantial fixation of the three-dimensional shape of the acellular tissue product is a staged fixation, wherein a first-stage shape fixation of the three-dimensional shape is performed on the acellular tissue product contained within the mold and a second-stage shape fixation is performed on the acellular tissue product following removal of the tissue product from the mold. For example, in some embodiments, the first-stage of fixation comprises subjecting the tissue product to heat under a reduced pressure or vacuum, the tissue product is removed from the mold, and the second-stage fixation of the three-dimensional shape is performed by exposing the tissue product having a three-dimensional shape to an electron beam.

After removal of the acellular tissue matrix from the mold and completion of all treatments to substantially fix the three-dimensional shape of the acellular tissue product, additional shaping may be performed. For example, a rim or other protrusion of tissue product attached to the substantially fixed, three-dimensional shape after removal from the mold may be removed by sharp cutting, such as by using a scalpel or scissors. In some embodiments, the trimming or shaping is performed by an automated device. In some embodiments, shaping or trimming can be performed manually, such as by the operating surgeon implanting the product, for example.

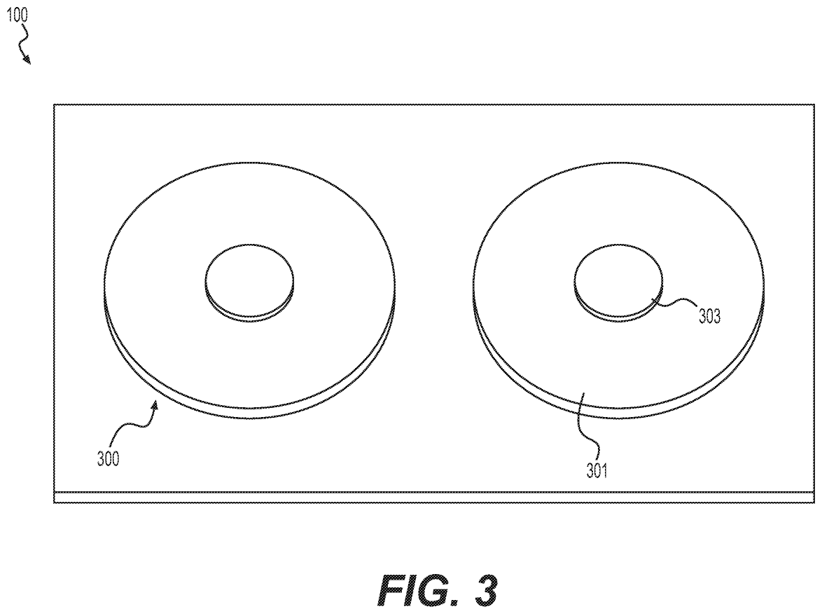

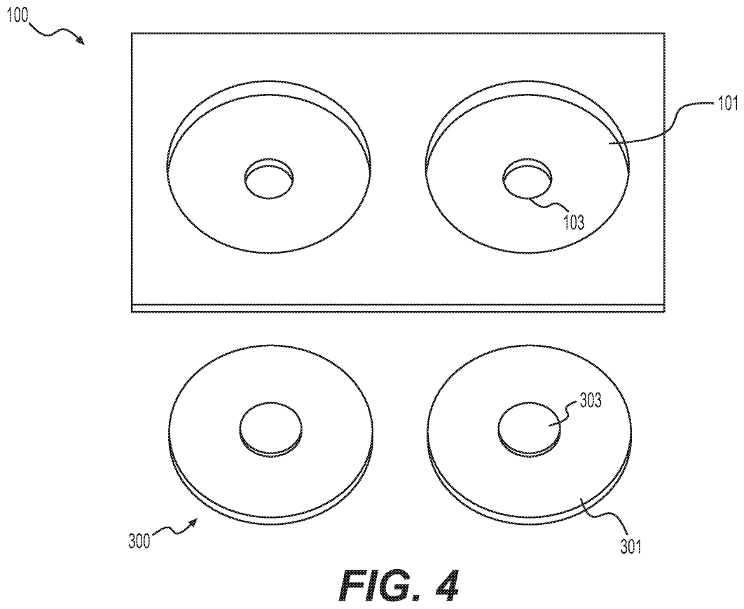

FIGS. 3 and 4 are exemplary depictions of a tissue product 300 with a substantially fixed shape. The tissue product 300 is shown within the mold 100 in FIG. 3 and is shown removed from the mold 100 in FIG. 4. The tissue product 300 includes a base 301 formed within the top reservoir 101. The base 301 corresponds to an areola of a selected tissue site. The tissue product 300 also includes a protrusion 303 formed with the bottom reservoir 103. The protrusion 301 corresponds to the nipple of a selected tissue site. Protrusion 303 may include material both above and below the plane of the base 301. In some embodiments, portions of the protrusion 303 may be removed after substantial fixation as described above.

The substantially fixed tissue product 300 may be packaged after fixation. For example, the tissue product 300 may be packaged in a blister pack or sealed in a plastic pouch. In some embodiments, the mold 100 is packaged with the tissue product 300. In some embodiments, the mold 100 and tissue product 300 are packaged prior to fixation.

In some embodiments, the tissue product 300 may be stored in a freeze-dried state. In some embodiments, the tissue product 300 may be stored in a hydrated state, or a state that does not require substantial rehydration or washing to remove storage components. In some embodiments, the tissue product 300 is stored in packaging as described above.

Implantation of Tissue Products

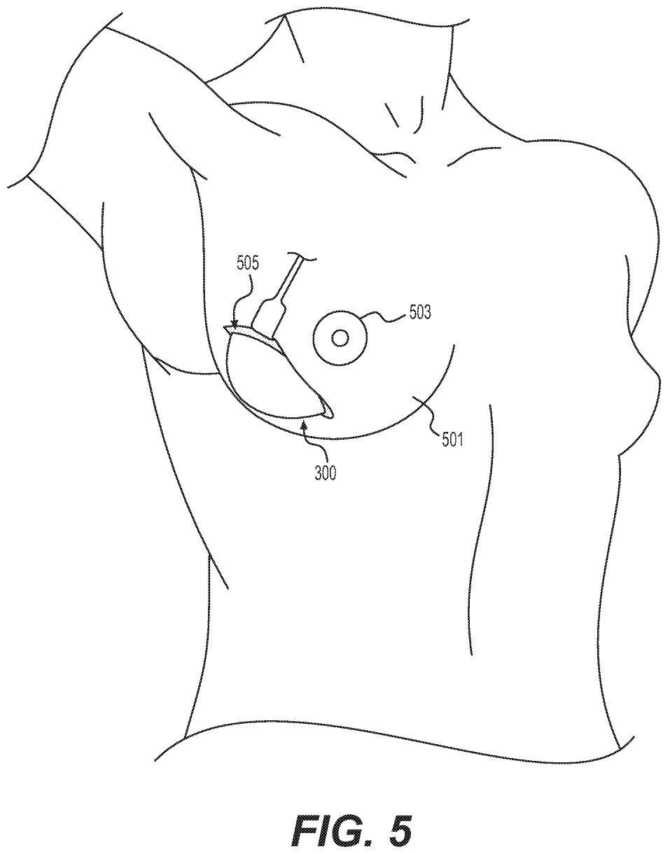

The tissue products produced according to the methods described above may be implanted into a patient. FIG. 5 depicts a tissue product implanted within a tissue site.

In some embodiments, the tissue product 300 is implanted in a breast 501. The tissue product 300 may be implanted as part of a breast reconstruction procedure, may be implanted following a mastectomy, or may be implanted concurrent with a breast implant or tissue expander. The breast may include, or formerly have included, a nipple-areolar complex 503.

In some embodiments, an incision 505 is made into a breast 501. The tissue product 300 is inserted into the incision 505. The tissue product 300 is oriented such that protrusion 303 faces outward from the body. The protrusion 303 is placed in a desired area on breast 501 to emulate a nipple-areolar complex. In some embodiments, further incisions may be made to the breast area including, or formerly including, the nipple-areolar complex 503 to create an opening to insert the protrusion 303 from within the breast 501.

Composite Tissue Products

In some embodiments, starting materials for forming a dermal appendage having a fixed three-dimensional shape include an acellular adipose matrix or an acellular muscle matrix. In some embodiments, the starting materials include a composite matrix including a combination of acellular dermal matrix, acellular adipose matrix, and/or acellular muscle matrix. The dermal, adipose, and muscle matrices may be mixed together or provided separately then combined to form a composite tissue product.

In some embodiments, the composite tissue product may include a mixture of multiple acellular tissue matrix materials such as dermal, adipose, muscle, or other suitable acellular tissue matrix material. In some embodiments, the composite tissue product may include acellular adipose tissue matrix surrounding one or more types of non-adipose acellular tissue matrix. In some embodiments, the composite tissue product may include a base and a protrusion composed of acellular tissue matrix compositions that vary from one another. For example, the protrusion may be composed of acellular dermal tissue matrix and the base may be composed of acellular dermal tissue matrix and acellular adipose tissue matrix. Alternatively, the composite can include a lattice or supporting structure produced from one tissue type and having pores or openings filled with tissue matrix of another type.

In some embodiments, the composite tissue product may include layers of acellular tissue matrix. Each layer of acellular tissue matrix may be composed of one or more types of acellular tissue matrix. For example, a layer may be composed of acellular dermal tissue matrix, acellular adipose tissue matrix, acellular muscle matrix, or a combination thereof. A composite tissue product may include one or more similar layers, alternating layers, or any combination of layers. In some embodiments, a top layer will be attached to a protrusion element.

In some embodiments, the composite tissue matrix may include a core of acellular tissue matrix surrounded by a sheath of acellular tissue matrix. The core or the sheath of acellular tissue matrix may be composed of one or more types of acellular tissue matrix. For example, the core may be composed of acellular dermal tissue matrix, acellular adipose tissue matrix, acellular muscle matrix, or a combination thereof and the sheath may be composed of acellular dermal tissue matrix, acellular adipose tissue matrix, acellular muscle matrix, or a combination thereof. A composite tissue product may include one or more layers of sheaths surrounding the core. In some embodiments, a top sheath will be attached to a protrusion element.

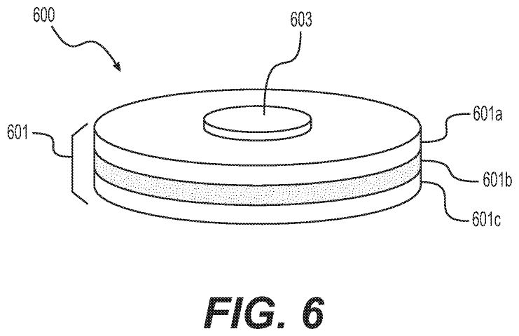

FIG. 6 depicts an exemplary composite tissue product 600. The composite tissue product may include a base 601 composed of multiple layers 601a, 601b, and 601c. The exemplary product depicted in FIG. 6 includes three layers but a composite tissue product as discussed herein may include any number of layers.

Layers 601a and 601c may be composed of acellular adipose tissue matrix and layer 601b may be composed of acellular dermal tissue matrix. In such a configuration, dermal layer 601b provides structural support to adipose layers 601a and 603c. Product 600 further includes a protrusion 603 primarily composed of adipose tissue matrix. The composite base 601 may act as a substrate, providing structural integrity for the adipose protrusion 603. Composite tissue products may be provided in different structural forms and may incorporate matrix material other than dermal or adipose matrix material.

### Example 1—Production of Ground Tissue Particles

Porcine acellular dermal matrix (pADM) was utilized to produce ground pADM particles. Tissue was washed in PBS twice prior to particle size reduction.

After PBS treatment, the pADM was coarsely ground in a commercial grinder. A range of sized plates may be used to coarsely ground the pADM. After coarse grinding, the pADM was finely ground in a second grinder. Various different plates may be used to finely ground the pADM. PBS was added during each grinding step to ensure all tissue was washed through grinding areas and minimal thermal damage to tissue occurred during the grinding steps. To remove excess PBS, the tissue slurry produced from grinding was centrifuged. The separated PBS solution was poured off and the remaining solid tissue was mixed until uniform and stored in a freezer until further processing.

### Example 2—Treatment of Ground Tissue Particles

The tissue produced in Example 1 was thawed and placed in processing bottles. A proteolytic solution was added to each bottle and mixed until it formed a uniform suspension. Then additional proteolytic solution was added and each bottle was shaken. The bottles were then centrifuged to remove the proteolytic solution. The tissue was then washed with PBS and centrifuged to remove the PBS solution. The PBS wash was then repeated, followed by another centrifuge step.

Then a DNase solution was added to each bottle and mixed until it formed a uniform suspension. Then an additional DNase solution was added and each bottle was shaken. The bottles were then centrifuged to remove the DNase solution. The tissue was then washed with PBS and centrifuged to remove the PBS solution. The PBS wash was then repeated, followed by another centrifuge step.

Then a diluted peracetic acid (PAA) solution was added to each bottle and mixed until it formed a uniform suspension. Then a diluted PAA solution was added and each bottle was shaken. The bottles were then centrifuged to remove the PAA solution. A protective solution was then added to the tissue and the bottles were mixed. The protective solution treatment was repeated multiple times.

Next the bottles were centrifuged. Excess solution was poured off and the tissue pellets let behind were mixed until uniform tissue slurry was formed. After this step, the percent of solids in the uniform slurry were measured. Then slurry was returned to the bottles.

Finally, the tissue slurry was treated with a protective storage solution and mixed until it formed a uniform suspension. Then an additional protective solution was added and each bottle was shaken. Then the bottles were centrifuged to remove the protective solution. Again, tissue pellets were left behind were mixed until uniform tissue slurry was formed.

After treatment with the protective solution, the percent of solids in the uniform slurry were measured again. Slurry samples containing more than a desired percentage of solids were treated again with the protective solution to reduce the percent solids.

### Example 3—Production of Nipple Shaped Tissue Products

The tissue slurry produced in Example 2 was placed into molds shaped similar to a nipple-areolar complex. The molded slurry was then exposed to e-beam radiation. After radiation, the resultant tissue products retained the nipple-areolar shape and would return to the nipple-areolar shape after slight compression.

The above description and embodiments are exemplary only and should not be construed as limiting the intent and scope of the invention.

What is claimed is:

1. A tissue product comprising:
a plurality of acellular tissue matrix particles formed into a three-dimensional shape, wherein the three-dimensional shape is in the shape of an anatomic structure, and wherein the three-dimensional shape is substantially fixed by irradiation.

2. The tissue product of claim 1, wherein the acellular tissue matrix particles comprise acellular dermal matrix.

3. The tissue product of claim 1, wherein the acellular tissue matrix particles comprise acellular adipose matrix.

4. The tissue product of claim 1, wherein the acellular tissue matrix particles comprise porcine-derived tissue matrix.

5. The tissue product of claim 1, wherein the acellular tissue matrix particles comprise human-derived tissue matrix.

6. The tissue product of claim 1, wherein the three-dimensional shape is formed by molding, electron beam exposure, dehydrothermal therapy, or a combination thereof.

7. The tissue product of claim 1, wherein the three-dimensional shape includes at least a first shape and a second shape.

8. The tissue product of claim 1, wherein the anatomical structure comprises a nipple.

9. The tissue product of claim 1, wherein the anatomic structure replicates an anatomic structure of a specific individual.

10. A method of forming a tissue product with a fixed three-dimensional shape comprising:
forming a plurality of acellular tissue matrix particles to a mold with a three-dimensional shape, wherein the three-dimensional shape is in the shape of an anatomic structure;
shaping the plurality of acellular tissue matrix particles within the mold into a stable, three-dimensional structure; and
substantially fixing the structure of the shaped plurality of acellular tissue matrix particles with irradiation.

11. The method of claim 10, wherein the acellular tissue matrix particles comprise acellular dermal matrix.

12. The method of claim 11, wherein the acellular dermal matrix particles comprise human acellular dermal matrix.

13. The method of claim 11, wherein the acellular dermal matrix particles comprise porcine acellular dermal matrix.

14. The method of claim 10, wherein the anatomic structure comprises a nipple.

15. The method of any of claim 10, wherein the mold is a reverse mold of the anatomic structure.

16. The method of any of claim 10, wherein the mold is a one-piece mold.

17. The method of any of claim 10, wherein the mold is at least a two-piece mold.

18. The method of any of claim 10, wherein the forming step comprises injecting the particles into the mold.

19. The method of any of claim 10, wherein the irradiation comprises electron beam radiation.

20. The method of any of claim 10, further comprising treating the particles contained within the mold with dehydrothermal therapy.
